(19) United States
(12) Patent Application Publication
(10) Pub. No.: US 2008/0008751 A1
(43) Pub. Date: Jan. 10, 2008

(54) Stable formulation comprising a combination of a moisture sensitive drug and a second drug and manufacturing procedure thereof

(76) Inventor: Michael Fox, Tel-Aviv (IL)

Correspondence Address: KENYON & KENYON LLP, ONE BROADWAY, NEW YORK, NY, 10004, US

(21) Appl. No.: 11/484,487

(22) Filed: Jul. 10, 2006

(51) Int. Cl. A61K 9/20; A61K 31/549

(52) U.S. Cl. 424/464; 514/223.5

(57) ABSTRACT

The present invention provides stable pharmaceutical compositions comprising a combination of active pharmaceutical ingredients. The pharmaceutical composition of the present invention comprises a moisture sensitive drug, in particular an angiotensin converting enzyme (ACE) inhibitor such as Cilazapril, as an active ingredient, a second pharmaceutically active ingredient such as for example Hydrochlorothiazide, and at least one pharmaceutical excipient, wherein the moisture sensitive active pharmaceutical ingredient is wet granulated with a solution of at least one pharmaceutical excipient, and methods for preparing such stable pharmaceutical compositions.

### FIELD OF THE INVENTION

The present invention relates to stable pharmaceutical compositions comprising a combination of a moisture sensitive active pharmaceutical ingredient (drug), in particular an angiotensin converting enzyme (ACE) inhibitor such as Cilazapril, and a second drug, such as Hydrochlorothiazide, as the active ingredients and methods for preparing such stable pharmaceutical compositions.

### BACKGROUND OF THE INVENTION

Cilazapril is apparently an angiotensin converting enzyme (“ACE”) inhibitor, which enzyme inhibits the formation of angiotensin II from angiotensin I by inhibiting the angiotensin converting enzyme. Chemically, Cilazapril is reported to be (1S,9S)-9-[(S)-1-Ethoxycarbonyl-3-phenylpropylamino]-10-oxoperhydropyridazino[1,2-a][1,2]diazepine-1-carboxylic acid and is understood to be disclosed in U.S. Pat. No. 4,512,924. Cilazapril has been prescribed in treating patients suffering from hypertension. Cilazapril has the following general formula:

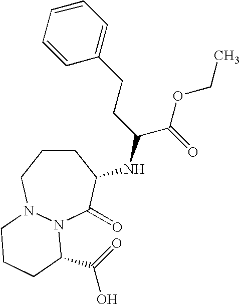

Hydrochlorothiazide is apparently a diuretic and an antihypertensive. Chemically, Hydrochlorothiazide, a 3,4-dihydro derivative of chlorothiazide, is reported to be 6-chloro-3,4-dihydro-2H-1,2,4-benzothiazidine-7-sulfonamide 1,1-dioxide and has the following general formula.

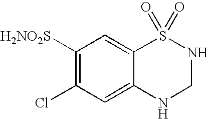

Hydrochlorothiazide is indicated as adjunctive therapy in edema associated with congestive heart failure, hepatic cirrhosis, and corticosteroid and estrogen therapy. It has also been found useful in edema due to various forms of renal dysfunction such as nephrotic syndrome, acute glomerulonephritis, and chronic renal failure. Further, Hydrochlorothiazide is indicated in the management of hypertension either as the sole therapeutic agent or to enhance the effectiveness of other antihypertensive drugs in the more severe forms of hypertension.

One of the requirements for an acceptable pharmaceutical composition is that it must be stable. A stable pharmaceutical composition does not exhibit substantial decomposition of the active pharmaceutical ingredient during the time between the manufacture of the composition and its use by a patient. Cilazapril and a number of other drugs suffer from instability problems because the active pharmaceutical ingredient rapidly degrades in the presence of water/moisture. Such active pharmaceutical ingredients (drugs) can therefore be characterized as moisture-sensitive drugs.

It is known that, tablet blends may be dry mixed, dry-granulated or wet-granulated before tableting. The choice of the processing procedure, dry mixing, dry granulation, wet granulation, or some other granulation process, depends on the properties of the drug and the chosen excipients. Generally, a dry manufacturing process is thought to be preferable for moisture-sensitive drugs.

To improve the stability of moisture sensitive drugs, water scavenger compounds may be incorporated into a tablet matrix. One such a water scavenger compound is the binder Copovidone (Plasdone S-630®), which binder is specifically recommended for moisture sensitive drugs. However, with very little success attempts were made to formulate Cilazapril tablets using this material in a dry granulation process. In such Cilazapril tablets degradation of the active pharmaceutical ingredient was apparent.

Wet-granulation processes have not been considered appropriate for moisture sensitive drugs since the very nature of these processes can include the presence of water/moisture. However, as described in copending U.S. application Ser. No. 11/446,336, filed Jun. 2, 2006, the best stability results can be achieved with a composition or formulation comprising the moisture sensitive drug and a binder such as Copovidone, wherein the formulation/composition is prepared using a wet granulation process, comprising wetting and then drying the composition at an elevated temperature.

Pharmaceutical compositions of such moisture sensitive active pharmaceutical ingredients (active drug substances) may contain one or more additional drug substances in a combination pharmaceutical composition. Such combination pharmaceutical compositions could provide enhanced treatment effectiveness or provide treatment while ameliorating undesired side effects of one such moisture sensitive active pharmaceutical ingredient. However, the combination of active pharmaceutical ingredients in one composition requires that the active pharmaceutical ingredients are compatible in terms of activity, side effects, and effectiveness for example.

Hydrochlorothiazide (HCTZ) can be used in combination with other anti-hypertensives. Moreover, pharmaceutical compositions for use in the treatment of hypertension may comprise a combination of Hydrochlorothiazide and an antihypertensive agent. Furthermore, HCTZ is compatible with the moisture sensitive active pharmaceutical ingredient Cilazapril for inclusion in a combination pharmaceutical composition. However, a pharmaceutical composition comprising a moisture sensitive drug substance and a second drug substance prepared using a single wet granulation process of the combined active ingredients appeared not to be a stable pharmaceutical composition but showed degradation of Cilazapril.

Surprisingly, the best stability results can be achieved, if a wet granulation process for preparing the combination pharmaceutical composition will be divided in at least two steps, wherein the second drug substance, preferably Hydrochlorothiazide (HCTZ), is added to the wet granulate after the wet granulation of the moisture sensitive drug substance, preferably Cilazapril, is completed.

### SUMMARY OF THE INVENTION

The present invention provides stable pharmaceutical compositions of a combination of Cilazapril and Hydrochlorothiazide (HCTZ), and methods of their preparation.

In one aspect the present invention provides a stable pharmaceutical composition comprising;

a) a moisture sensitive active pharmaceutical ingredient; and

b) a second active pharmaceutical ingredient;

wherein the moisture sensitive active pharmaceutical ingredient is first wet granulated with a solution of at least one pharmaceutical excipient in at least one processing solvent before granulation with the second active pharmaceutical ingredient. Preferably, at least one excipient is a binder.

In another aspect the present invention provides a method of preparing a combined granular composition comprising a wet granulated moisture sensitive active pharmaceutical ingredient and a second active pharmaceutical ingredient comprising the following steps of

a) providing a moisture sensitive active pharmaceutical ingredient;

b) mixing the moisture sensitive active pharmaceutical ingredient with at least one pharmaceutically acceptable excipient other than a binder, forming a mixture; and

c) wet granulating the mixture with a solution of a binder excipient dissolved in one or more processing solvents forming a wet granulate;

d) providing a material comprising a second active pharmaceutical ingredient and optionally one or more pharmaceutical excipients; and

e) adding the material from step d) to the wet granulate from step c) forming a combined granulate,

wherein when the material of step d) comprises a second pharmaceutical ingredient and one or more pharmaceutical excipients the material is optionally a mixture obtained by mixing the second pharmaceutical ingredient with the one or more pharmaceutical excipient.

In another embodiment of the present invention the method further comprises steps of preparing a tablet pharmaceutical composition of the present invention wherein the method further comprises the steps of

f) mixing the combined granulate with one or more excipients forming a final blend;

g) pressing the final blend into a tablet; and

h) optionally coating the tablet with a cosmetic coat.

The present invention also provides a method of treating a patient suffering from a disease, preferably hypertension, comprising administering to a patient in need thereof a therapeutically effective amount of a stable pharmaceutical composition comprising a moisture sensitive active pharmaceutical ingredient, preferably Cilazapril, a second active pharmaceutical ingredient, preferably Hydrochlorothiazide, and at least one pharmaceutical excipient, wherein the active pharmaceutical ingredients are wet granulated with a solution of the at least one pharmaceutical excipient.

### DETAILED DESCRIPTION OF THE INVENTION

As used herein the term moisture sensitive active pharmaceutical ingredient refers to an active pharmaceutical ingredient which rapidly degrades in the presence of water/moisture. In understanding the term “moisture sensitive active pharmaceutical ingredient” it is helpful to consider how the exemplified Cilazapril behaves on storage. When commercially available Cilazapril tablets (Vascace®) are stored at 55° C. and 75% relative humidity in the marketed package, “aluminum-aluminum cold form blisters”, which are considered the “gold standard” in blister packaging with respect to moisture barrier blister packs, the degradation as evidenced by formation of Cilazaprilat is of the order of one percent or so over the initial Cilazaprilat content as compared with the initial Cilazapril content, over a seven day storage period, whereas when the tablets are removed from this package and stored at the same conditions and thus exposed to moisture, for just 48 hours, greater than five times of the Cilazaprilat formation is observed, see Table 1 below. It is this type of characteristic that identifies Cilazapril as a moisture sensitive active pharmaceutical ingredient.

TABLE 1

| | | Storage period, | Total Degradation |
|---|---|---|---|
| Lot No | Storage conditions | hours | Products, % |
| # B2017 | Packed | Time “0” | 1.1 |
| # B2017 | Packed | 168 | 2.1 (1%) |
| # B2017 | Packed | 336 | 2.8 (1.7%) |
| # B2017 | Unpacked | 48 | 7.5 (6.4%) |

Wet-granulation processes have not been considered appropriate for moisture sensitive drugs since the very nature of these processes can include the presence of water/moisture. However, as described in copending U.S. application Ser. No. 11/446,336, filed Jun. 2, 2006, the best stability results can be achieved with a composition or formulation comprising the moisture sensitive drug and a binder such as Copovidone, wherein the formulation/composition is prepared using a wet granulation process, comprising wetting and then drying the composition at an elevated temperature.

Pharmaceutical compositions of such moisture sensitive active pharmaceutical ingredients (active drug substances) may contain one or more additional drug substances in a combination pharmaceutical composition. Such combination pharmaceutical compositions could provide enhanced treatment effectiveness or provide treatment while ameliorating undesired side effects of one such moisture sensitive active pharmaceutical ingredient. However, the combination of active pharmaceutical ingredients in one composition requires that the drug substances are compatible in terms of activity, side effects, and effectiveness for example. A compatible second drug substance for inclusion in a combination pharmaceutical composition comprising the moisture sensitive drug substance Cilazapril, is for example Hydrochlorothiazide (HCTZ).

However, a pharmaceutical composition comprising a moisture sensitive drug substance and a second drug substance using wet granulation of the combined drug substances appeared not to be a stable pharmaceutical composition. In particular, such pharmaceutical composition containing only Cilazapril as its active pharmaceutical ingredrient was shown to be stable but showed unacceptable degradation to Cilazaprilat, when Cilazapril was combined with a second drug substance such as HCTZ in a composition where a combined mixture of the drug substances was wet granulated.

Surprisingly, the best stability results can be achieved, if a wet granulation process for preparing the combination pharmaceutical composition will be divided in at least two steps, wherein the second drug substance, preferably Hydrochlorothiazide (HCTZ), is added to the wet granulate after first the granulation of the moisture sensitive drug substance, preferably Cilazapril, is completed. HCTZ can be added to the wet granulate alone or together with other ingredients and/or in a granulation solution.

In one aspect the present invention provides a pharmaceutical composition comprising;

a) a moisture sensitive active pharmaceutical ingredient, preferably Cilazapril; and

b) a second active pharmaceutical ingredient, preferably Hydrochlorothiazide;

wherein the moisture sensitive active pharmaceutical ingredient is first wet granulated with at least one pharmaceutical excipient in at least one processing solvent before granulation with the second active pharmaceutical ingredient. Compositions of this aspect of the invention are stable.

In the context of the present invention, the first wet granulation step should preferably not involve a significant proportion of the second active pharmaceutical ingredient, and most preferably does not contain any of the second active pharmaceutical ingredient. Preferably, at least one excipient is a binder and the pharmaceutical composition comprises at least two pharmaceutical excipients.

Preferably the amount of the moisture sensitive active pharmaceutical ingredient in the composition is about 0.1% to about 25%, more preferably about 0.5% to about 15%, of the total weight of the composition. A most preferred amount of the moisture sensitive active pharmaceutical ingredient in the composition is about 0.6% to about 2.7% of the total weight of the composition. Preferably, the moisture sensitive active pharmaceutical ingredient is Cilazapril.

Preferably the amount of the second active pharmaceutical ingredient in the composition is about 1% to about 25%, more preferably of about 3% to about 15%, of the total weight of the composition. A most preferred amount of the second active pharmaceutical ingredient in the composition is about 5% to about 10% of the total weight of the composition. Preferably, when the moisture sensitive active pharmaceutical ingredient is an “ACE inhibitor”, the second active pharmaceutical ingredient is preferably a diuretic drug. More preferably a thiazide derivative. Most preferably Hydrochlorothiazide (HCTZ).

The present invention further provides a stable pharmaceutical composition comprising a moisture sensitive active pharmaceutical ingredient, a second active pharmaceutical ingredient and at least one pharmaceutically acceptable excipient, wherein the composition contains not more than 3% (w/w of the initial amount of the moisture sensitive active pharmaceutical ingredient) of a major degradation product after storage in a package with moisture barrier properties which properties are at least as efficient as aluminum-aluminum cold form blisters. Preferably, the concentration of the major degradation product in the stable pharmaceutical composition of the present invention after storage as described above is not more than 2%. More preferably, the concentration of the major degradation product in the stable pharmaceutical composition of the present invention after storage as described above is not more than 1%. Storage may comprise storage at a temperature of 55° C. for 14 days and storage at a temperature of 40° C. and 75% relative humidity for three months. The degradation product may be detected by HPLC analysis. Preferably, the moisture sensitive active pharmaceutical ingredient is Cilazapril and the degradation product is its major degradation product Cilazaprilat. Preferably, the second pharmaceutical ingredient is HCTZ.

A stable pharmaceutical composition of the present invention therefore provides a pharmaceutical composition of a moisture sensitive active pharmaceutical ingredient and a second pharmaceutical ingredient, preferably Cilazapril and HCTZ respectively, characterized by comprising not more than 3%, preferably not more than 2%, most preferably not more than 1%, by weight per weight of the total amount of moisture sensitive pharmaceutical ingredient, Cilazapril, of its major degradation Cilazaprilat product upon storage.

It is understood that Cilazaprilat has the following structure:

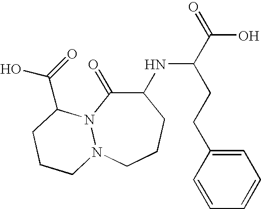

Preferably, the stable pharmaceutical composition of the present invention comprises at least about 4% of a binder by total weight of the composition. Preferably, the pharmaceutical composition comprises from about 4% to about 20%, more preferably from about 5% to about 10% of a binder by total weight of the composition. The binder comprises for example, one or more of, a cellulose derivative, a polyvinyl pyrrolidone (PVP) and its derivatives, a polyvinylacetate (PVA) or a polyvinyl alcohol. Examples of suitable cellulose derivatives as a binder in the present invention are Hydroxypropylmethyl cellulose (HPMC) or Hydroxypropyl cellulose (HPC). More preferably, the binder is the Copovidone, exemplified by Plasdone® S-630 (Copovidone), which is a synthetic, 60:40, linear, random copolymer of N-vinyl-2-pyrrolidone and vinyl acetate, and which has a reduced hydrophilicity and a reduced polymer glass transition temperature (Tg) in comparison to a polyvinyl pyrrolidone (PVP) homopolymer. In the stable pharmaceutical composition of the present invention this binder is wet granulated with the moisture sensitive active pharmaceutical ingredient and one or more pharmaceutical excipients in a processing solvent to form a wet granulate before granulating the wet granulate with a second drug substance.

The stable pharmaceutical compositions comprising a moisture sensitive active pharmaceutical ingredient and a second drug substance of the present invention may further contain excipients such as tablet and capsule fillers and diluents (such as microcrystalline cellulose, lactose, starch and tri-basic calcium phosphate), disintegrants (such as starch, croscarmellose sodium, crospovidone and sodium starch glycolate), and glidants (such as colloidal silicon dioxide and talc), lubricants (such as magnesium stearate, sodium lauryl sulfate, stearic acid and sodium stearyl fumarate).

More particularly, suitable diluents and fillers for use in the pharmaceutical composition of the present invention include microcrystalline cellulose (e.g. Avicel®), lactose, starch, pregelatinized starch, calcium carbonate, calcium sulfate, sugar, dextrates, dextrin, dextrose, dibasic calcium phosphate dihydrate, tribasic calcium phosphate, magnesium carbonate, magnesium oxide, maltodextrin, mannitol, powdered cellulose, sodium chloride, sorbitol and talc.

Preferably, the pharmaceutical composition of the present invention includes lactose monohydrate, more preferably in an amount of about 50% to about 65%, most preferably of about 55% to about 60% by total weight of the composition.

In an alternative preferred embodiment, the pharmaceutical composition of the present invention includes talc, more preferably in an amount of about 1% to about 2% by total weight of the composition.

The pharmaceutical composition may also include both lactose monohydrate and talc in the amounts specified above.

Solid pharmaceutical compositions of the present invention that are compacted into a dosage form, such as a tablet, may include the addition of a disintegrant to the composition. Disintegrants include croscarmellose sodium (e.g. Ac Di Sol®, Primellose®), crospovidone (e.g. Kollidon®, Polyplasdone®), microcrystalline cellulose, polacrilin potassium, powdered cellulose, pregelatinized starch, sodium starch glycolate (e.g. Explotab®, Primoljel®) and starch.

Preferably, the pharmaceutical composition of the present invention includes starch, more preferably in an amount of about 20% to about 30%, most preferably about 25% by total weight of the composition.

Glidants can be added to improve the flowability of a solid composition before compaction and to improve the accuracy of dosing especially during compaction and capsule filling. Excipients that may function as glidants include colloidal silicon dioxide, magnesium trisilicate, powdered cellulose, and talc.

A lubricant can be added to the composition to reduce adhesion and/or ease the release of the product from e.g. the dye. Lubricants include magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc and zinc stearate.

Preferably, the pharmaceutical composition of the present invention includes sodium stearyl fumarate, more preferably in an amount of about 0.5% to about 1.5%, most preferably about 1% by total weight of the composition.

Other excipients that may be incorporated into the formulation include preservatives, surfactants, antioxidants, or any other excipient commonly used in the pharmaceutical industry.

In a preferred embodiment of the present invention, the stable formulation comprises Cilazapril, HCTZ, copovidone, lactose monohydrate, sodium starch glycolate, talc extra fine and sodium stearyl fumarate. Preferably, the pharmaceutical composition comprises (by total weight of the composition), Cilazapril in an amount of about 0.5% to about 15%, more preferably of about 0.6% to about 2.7%, HCTZ in an amount of about 3% to about 15%, more preferably of about 5% to about 10%, lactose monohydrate in an amount of about 50% to about 65%, more preferably of about 55% to about 60%, talc in an amount of about 1% to about 2%, starch in an amount of about 20% to about 30%, more preferably about 25%, a binder, preferably copovidone, in an amount of about 4% to about 20%, more preferably 5% to 10%, and sodium stearyl fumarate in an amount of about 0.5% to about 1.5%, more preferably about 1%.

The solid compositions of the present invention include powders, granulates, aggregates and compacted compositions. The dosages include dosages suitable for oral, buccal, and rectal administration. Although the most suitable administration in any given case will depend on the nature and severity of the condition being treated, the most preferred route of the present invention is oral. The dosages may be conveniently presented in unit dosage form and prepared by any of the methods well known in the pharmaceutical arts.

The pharmaceutical composition of the present invention may be prepared in any dosage form such as a compressed granulate in the form of a tablet for example. Also, uncompressed granulates and powder mixes that are obtained by the method of the present invention in the pre-compression steps can be simply provided in a dosage form of a capsule or sachet. Therefore, dosage forms of the pharmaceutical composition of the present invention include solid dosage forms like tablets, powders, capsules, sachets, etc. The dosage form of the present invention may also be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

Once a solid composition comprising a moisture sensitive active pharmaceutical ingredient, preferably Cilazapril, and a second drug substance, preferably HCTZ, is prepared in accordance with the present invention, it is preferably formulated into pharmaceutical formulations such as conventional dosage forms, including tablets and capsules. Tablets are a preferred dosage form. In addition, the tablets may be coated with an optional cosmetic tablet coating. More preferably this cosmetic coat has “moisture barrier” properties. This moisture barrier property provides protection against environmental moisture for sensitive cores, enhances product stability, and improves shelf life. Preferably, the cosmetic coating is a tablet coating based on polyvinyl alcohol. More preferably, the cosmetic coating comprises polyvinyl alcohol, talc and polyethylene glycol (PEG). Most preferably, the cosmetic coating further comprises an opacifier and/or a colorant, e.g. titanium dioxide and/or iron oxide.

The commercially available series of powder mixes for coating suspension sold as the Opadry®II 85F series (a coating with moisture barrier properties), available from Colorcon, which are based on Polyvinyl alcohol, are examples of such cosmetic coat. In addition to Polyvinyl Alcohol, this Opadry series of products comprise Talc, PEG 3350, Titanium Dioxide and pigments. Preferably, the tablets of the present invention comprises a cosmetic coat of about 2% to about 6% of the tablet weight, more preferably of about 2.5% to about 4.5% of the tablet weight, most preferably of about 3% to about 3.5% of the tablet weight.

In another embodiment the present invention provides a method of preparing a pharmaceutical composition comprising a moisture sensitive active pharmaceutical ingredient and a second active pharmaceutical ingredient comprising the following steps of

a) providing a moisture sensitive active pharmaceutical ingredient;

b) mixing the moisture sensitive active pharmaceutical ingredient with at least one pharmaceutically acceptable excipient, forming a mixture; and

c) wet granulating the mixture with a solution of a binder excipient dissolved in one or more processing solvents forming a wet granulate;

d) providing a material comprising a second active pharmaceutical ingredient and optionally one or more pharmaceutical excipients; and

e) adding the material from step d) to the wet granulate from step c) forming a combined granulate,

wherein when the material of step d) comprises a second pharmaceutical ingredient and one or more pharmaceutical excipients the material is optionally a mixture obtained by mixing the second pharmaceutical ingredient with the one or more pharmaceutical excipients.
Preferably, the moisture sensitive active pharmaceutical ingredient is Cilazapril and the second active pharmaceutical ingredient is Hydrochlorothiazide (HCTZ). In a preferred embodiment, the pharmaceutical excipient(s) employed in step b) do not include a binder.

Preferably the amount of the moisture sensitive active pharmaceutical ingredient in the composition is about 0.1% to about 25%, more preferably of about 0.5% to about 15%, of the total weight of the composition. A most preferred amount of the moisture sensitive active pharmaceutical ingredient in the composition is about 0.6% to about 2.7% of the total weight of the composition. Preferably, the moisture sensitive active pharmaceutical ingredient is Cilazapril.

Preferably the amount of the second active pharmaceutical ingredient in the composition is about 1% to about 25%, more preferably of about 3% to about 15%, of the total weight of the composition. A most preferred amount of the second active pharmaceutical ingredient in the composition is about 5% to about 10% of the total weight of the composition. Preferably, when the moisture sensitive active pharmaceutical ingredient is an ACE inhibitor, the second active pharmaceutical ingredient is Hydrochlorothiazide (HCTZ).

In preparing a pharmaceutical composition of the present invention a typical granulation process involves mixing the moisture sensitive active ingredient and possibly excipients in a mixer. The binder is dissolved in the processing solvent used for granulating although a further portion of binder or another binder may be one of the excipients added in the initial dry mix state with the moisture sensitive drug substance. The granulating/processing solvent, solution or suspension is added to the dry powders in the mixer and mixed to form a wet granulate. The second drug substance is added to the wet granulate either alone or with one or more excipients, optionally in a processing solvent, and mixed until the desired characteristics are achieved. This usually produces a granule that will have suitable characteristics for producing tablets with adequate hardness, dissolution, content uniformity, and other physical characteristics. After the wet granulation step, the product is most often dried and then milled after drying, to obtain a major percentage of the product within a desired size range. Preferably, the product after wet granulation is dried until the loss on drying (LOD) is not more than about 2.5%, more preferably not more than about 1.5%. Preferably, the product is milled or sized through a 1 mm aperture screen, more preferably through a 0.8 mm aperture screen.

Preferably, the stable pharmaceutical composition of the present invention is prepared by wet granulation with a suitable solvent/processing solvent. A suitable solvent/processing solvent is able to dissolve the selected binder. Preferably, the solvent/processing solvent is capable of dissolving the binder to reach a concentration of at least about 10% W/W. More preferably, the solvent/processing solvent is selected from the group consisting of ethanol, isopropyl alcohol, water, and combinations thereof. Preferably, the stable formulation prepared by wet granulation comprises at least 4%, preferably about 4% to about 20%, more preferably about 5% to about 10%, of a binder by weight of the formulation. Suitable binders for use in the method of the present invention include cellulose derivatives, polyvinyl pyrrolidones (PCP) and its derivatives, polyvinylacetates (PVA), or polyvinylalcohols. Preferably, the binder comprises at least Copovidone and more preferably, the binder is applied as a solution in ethanol or water. A preferred solution of the binder in ethanol or water comprises about 25% to about 55% (w/w) binder, preferably Copovidone, more preferably about 30% to about 50% (w/w) binder, preferably Copovidone.

Moreover, the copending U.S. application Ser. No. 11/446,336, filed Jun. 2, 2006, incorporated herein by reference, further describes preparing a stable pharmaceutical composition comprising a moisture sensitive active pharmaceutical ingredient using wet granulation. A process of preparing a wet granulate comprising a moisture sensitive active pharmaceutical ingredient and a binder is described therein.

The method of the present invention may further comprise steps in preparing a tablet or capsule of the pharmaceutical composition of the present invention. In preparing such tablet the method further comprises the steps of

f) mixing the combined granulate from step e) with one or more excipients forming a final blend;

g) pressing the final blend into a tablet; and

h) optionally coating the tablet with a cosmetic coat. Preferably, the cosmetic coat has moisture barrier properties. Examples of such cosmetic coatings are tablet coatings based on polyvinyl alcohol.

The optional cosmetic coating of the tablet preferably comprises preparing a suspension comprising about 10% to about 25%, preferably about 12% to about 15%, more preferably about 12% to about 13%, of a powder mixture for cosmetic coating, and applying the suspension on the tablet. The cosmetic coating suspension is preferably prepared such that the tablet comprises about 2% to about 6%, preferably about 2.5% to about 4.5%, of a tablet cosmetic coat. The tablet cosmetic coat in the present invention preferably has “moisture barrier” properties. The commercially available series of powder mixes for coating suspension sold as the Opadry®II 85F series, available from Colorcon, which are based on Polyvinyl alcohol, are examples of such cosmetic coat with moisture barrier properties.

Capsules comprising either a hard or soft shell and containing the composition of the present invention may be prepared. The shell may be made from gelatin and optionally contain a plasticizer such as glycerin and sorbitol, and an opacifying agent or colorant. A capsule filling of the present invention may comprise the granulates that were described with reference to tableting, a final blend of a granulate composition of the present invention mixed with one or more excipients, however they are not subjected to a final tableting step. Further, such capsules may be prepared by any of the methods well known in the pharmaceutical arts.

Further, in a preferred embodiment, the present invention provides a method for preparing a stable pharmaceutical composition comprising:

a) mixing cilazapril, lactose, talc and starch;

b) adding a solution of a binder, preferably copovidone, to the mixture obtained in step a) to form a wet granulate;

c) optionally combining the wet granulate with further starch and mixing;

d) adding HCTZ to the wet granulate and mixing;

e) drying and then milling the granulate; and

f) adding sodium stearyl fumarate to the granulate obtained in step e) and mixing to obtain a final blend.

The compositions of the present invention are useful in therapy. In particular, the compositions of the invention are useful in treating hypertension.

The present invention also provides a method of treating a patient suffering from a disease, preferably hypertension, comprising administering to a patient in need thereof a therapeutically effective amount of a stable pharmaceutical composition comprising a moisture sensitive active pharmaceutical ingredient, preferably Cilazapril, a second active pharmaceutical ingredient, preferably Hydrochlorothiazide, and at least one pharmaceutical excipient, wherein the active pharmaceutical ingredients are wet granulated with a solution of at least one pharmaceutical excipient.

The following examples are presented in order to further illustrate the invention. These examples should not be construed in any manner to limit the invention.

### EXAMPLES

### Example 1

### Wet Granulation, Hypromellose (HPMC) as a Binder, One Step Granulation

In a high shear mixer were mixed for 1 minute: 7.8 g of Cilazapril Monohydrate, 178.8 g of Lactose Monohydrate, 4.5 g of Talc Extra Fine, 18.8 g of Hydrochlorothiazide and 75.0 g of Starch. 60 g of a 20% (w/w) aqueous solution of Hypromellose was added and the mass was mixed in the high shear mixer for 4 minutes. 6 g of water was added and the blend was mixed for 1 minute in the high shear mixer. The obtained granulate was dried using a fluid bed dryer and the dry granulate was milled in an oscillating granulator through 0.8 mm screen. The milled granulate was combined with 2.7 g of screened Sodium Stearyl Fumarate and mixed in a Y-cone blender for 5 minutes.

Tablets were pressed from the final blend in a rotary tablet press. The tablets were packed in cold formed aluminium blister covered with aluminium foil. Packed tablets were stored at 55° C. The main degradation product, Cilazaprilat, was tested using HPLC method.

### Example 2

### Wet Granulation, Copovidone as a Binder, One Step Granulation

In a high shear mixer were mixed for 1 minute: 5.2 g of Cilazapril Monohydrate, 115.3 g of Lactose Monohydrate, 3.0 g of Talc Extra Fine, 12.5 g of Hydrochlorothiazide and 50.0 g of Starch. 33 g of a 36.4% (w/w) aqueous solution of Copovidone was added and the mass was mixed in the high shear mixer for 3 minutes. The obtained granulate was dried using a fluid bed dryer and the dry granulate was milled in an oscillating granulator through 0.8 mm screen. The milled granulate was combined with 1.6 g of screened Sodium Stearyl Fumarate and mixed in a Y-cone blender for 5 minutes.

Tablets were pressed from the final blend in a rotary tablet press. The tablets were packed in cold formed aluminium blister covered with aluminium foil. Packed tablets were stored at 55° C. The main degradation product, Cilazaprilat, was tested using HPLC method.

### Example 3

### Wet Granulation, Hypromellose (HPMC) as a Binder, Two Steps Granulation

In a high shear mixer were mixed for 1 minute: 10.4 g of Cilazapril Monohydrate, 238.4 g of Lactose Monohydrate, 6.0 g of Talc Extra Fine, and 100.0 g of Starch. 80 g of a 20% (w/w) aqueous solution of Hypromellose was added and the mass was mixed in the high shear mixer for 3 minutes. 8.2 g of water was added and the blend was mixed for 1 minute in the high shear mixer. 25.0 g of Hydrochlorothiazide was added to the wet blend and the mass was mixed for 2 minutes in the high shear mixer. The obtained granulate was dried using a fluid bed dryer and the dry granulate was milled in an oscillating granulator through 0.8 mm screen. The milled granulate was combined with 3.8 g of screened Sodium Stearyl Fumarate and mixed in a Y-cone blender for 5 minutes.

Tablets were pressed from the final blend in a rotary tablet press. The tablets were packed in cold formed aluminium blister covered with aluminium foil. Packed tablets were stored at 55° C. The main degradation product, Cilazaprilat, was tested using HPLC method.

### Example 4

### Wet Granulations Copovidone as a Binders Two Steps Granulation

In a high shear mixer were mixed for 1 minute: 10.4 g of Cilazapril Monohydrate, 234.4 g of Lactose Monohydrate, 6.0 g of Talc Extra Fine, and 100.0 g of Starch. 66 g of a 36.4% (w/w) aqueous solution of Copovidone was added and the mass was mixed in the high shear mixer for 10 minutes. 25.0 g of Hydrochlorothiazide was added to the wet blend and the mass was mixed for 2 minutes in the high shear mixer. The obtained granulate was dried using a fluid bed dryer and the dry granulate was milled in an oscillating granulator through 0.8 mm screen. The milled granulate was combined with 3.9 g of screened Sodium Stearyl Fumarate and mixed in a Y-cone blender for 5 minutes.

Tablets were pressed from the final blend in a rotary tablet press. The tablets were packed in cold formed aluminium blister covered with aluminium foil.

Packed tablets were stored at 55° C. The main degradation product, Cilazaprilat, was tested using HPLC method.

### Example 5

### Wet Granulation, Copovidone as a Binder, Two Steps Granulation, Starch and Part of the Granulation Solution Added in Second Granulation Step

In a high shear mixer were mixed for 1 minute: 5.2 g of Cilazapril Monohydrate, 115.3 g of Lactose Monohydrate and 3.0 g of Talc Extra Fine. 27.5 g of a 36.4% (w/w) aqueous solution of Copovidone was added and the mass was mixed in the high shear mixer for 1.7 minutes. 50.0 g of Starch was added to the wet blend and the mass was mixed for 2 minutes in the high shear mixer. 12.5 g of Hydrochlorothiazide was added to the wet blend and the mass was mixed for 20 second in the high shear mixer. 5.5 g of 36.4% (w/w) aqueous solution of Copovidone was added and the mass was mixed in the high shear mixer for 2 minutes. The obtained granulate was dried using a fluid bed dryer and the dry granulate was milled in an oscillating granulator through 0.8 mm screen. The milled granulate was combined with 1.7 g of screened Sodium Stearyl Fumarate and mixed in a Y-cone blender for 5 minutes.

Tablets were pressed from the final blend in a rotary tablet press. The tablets were packed in cold formed aluminium blister covered with aluminium foil. Packed tablets were stored at 55° C. The main degradation product, Cilazaprilat, was tested using HPLC method.

TABLE 2
Examples and comparative examples of pharmaceutical
compositions comprising Cilazapril, a moisture sensitive active
pharmaceutical ingredient, and Hydrochlorothiazide.

| | Example | | | |
|---|---|---|---|---|
| | Ex. 1 and 3 | Ex. 2 | Ex. 4 | Ex. 5 |
| | Binder | | | |
| | HPMC | Copovidone | Copovidone | Copovidone |
| Ingredient | Content, % of the end tablet weight | | | |
| Cilazapril Monohydrate | 2.61 | 2.58 | 2.61 | 2.61 |
| Hydrochlorothiazide | 6.25 | 6.19 | 6.25 | 6.25 |
| Lactose Monohydrate | 59.59 | 58.01 | 57.64 | 57.64 |
| Talc Extra Fine | 1.50 | 1.49 | 1.50 | 1.50 |
| Starch | 25.00 | 24.75 | 25.00 | 25.00 |
| Hypromellose | 4.00 | | | |
| Copovidone | | 5.94 | 6.00 | 6.00 |
| Sodium Stearyl Fumarate | 1.05 | 1.04 | 1.00 | 1.00 |
| Water (process solvent only) | 22.2/18.2 | 10.5 | 10.5 | 10.5 |

### Example 6

### Stability Testing of Pharmaceutical Compositions of Cilazapril and HCTZ

Comparative stability tests were performed comparing the stability of pharmaceutical compositions of Cilazapril and HCTZ prepared either in a process wet granulating a mixture of Cilazapril and HCTZ (a “single step wet granulation process”) with such pharmaceutical compositions prepared in a process wet granulating first Cilazapril forming a wet granulate to which the HCTZ is added (a “two step wet granulation process”). Table 3 shows the results of storage of such compositions (Examples 1-5) under stress conditions of 55° C. for 4 weeks. The results in the table shows that the two step wet granulation process of pharmaceutical compositions of Cilazapril and HCTZ of the present invention provides a stable pharmaceutical composition compared to the one step wet granulating process.

TABLE 3
Degradation under “stress” conditions (55° C., storage period 4 weeks),
of Cilazapril and HCTZ displayed as function of different formulations and
manufacturing methods.

| | One step | One step | Two steps | Two steps | Two steps |
|---|---|---|---|---|---|
| | granulation, | granulation, | granulation, | granulation, | granulation, |
| | HPMC as a | Copovidone as a | HPMC as a | Copovidone as a | Copovidone as |
| Description | binder (ex. 1) | binder (ex. 2) | binder (ex. 3) | binder (ex. 4) | a binder (ex. 5) |
| Major degradation | 0.3 | 0.2 | 0.1 | 0.1 | 0.1 |
| product, | | | | | |
| Cilazaprilat, Time | | | | | |
| “Zero”. | | | | | |
| % per labeled | | | | | |
| claim of Cilazapril | | | | | |
| Major degradation | 8.8 | 8.3 | 2.4 | 1.3 | 1.4 |
| product, | | | | | |
| Cilazaprilat, | | | | | |
| storage period | | | | | |
| 4 weeks, | | | | | |
| % per labeled | | | | | |
| claim of Cilazapril | | | | | |

1. A stable pharmaceutical composition comprising;
a) a moisture sensitive active pharmaceutical ingredient; and
b) a second active pharmaceutical ingredient;
wherein the moisture sensitive active pharmaceutical ingredient is first wet granulated with a solution of at least one pharmaceutical excipient in at least one processing solvent before granulation with the second active pharmaceutical ingredient.

2. The stable pharmaceutical composition according to claim 1, wherein the amount of the moisture sensitive active pharmaceutical ingredient is about 0.1% to about 25% of the total weight of the composition.

3. The stable pharmaceutical composition according to claim 2, wherein the amount of the moisture sensitive active pharmaceutical ingredient is about 0.5% to about 15% of the total weight of the composition.

4. The stable pharmaceutical composition according to claim 3, wherein the amount of the moisture sensitive active pharmaceutical ingredient is about 0.6% to about 2.7% of the total weight of the composition.

5. The stable pharmaceutical composition according to claim 1, wherein the moisture sensitive active pharmaceutical ingredient is Cilazapril.

6. The stable pharmaceutical composition according to claim 1, wherein the amount of the second active pharmaceutical ingredient is about 1% to about 25% of the total weight of the composition.

7. The stable pharmaceutical composition according to claim 6, wherein the amount of the second active pharmaceutical ingredient is about 5% to about 10% of the total weight of the composition.

8. The stable pharmaceutical composition according to claim 1, wherein the second active pharmaceutical ingredient is Hydrochlorothiazide.

9. The stable pharmaceutical composition according to claim 1, wherein at least one pharmaceutical excipient is a binder.

10. The stable pharmaceutical composition according to claim 9, wherein the binder is selected from the group consisting of cellulose derivatives, polyvinyl pyrrolidones and their derivatives, polyvinyl acetates, and polyvinyl alcohols.

11. The stable pharmaceutical composition according to claim 10, where the binder is selected from the group consisting of Copovidone and Hypromellose.

12. The stable pharmaceutical composition according to claim 9, wherein the amount of the binder is at least about 4% of the total weight of the composition.

13. The stable pharmaceutical composition according to claim 12, wherein the amount of the binder is about 4% to about 20% of the total weight of the composition.

14. The stable pharmaceutical composition according to claim 13, wherein the amount of the binder is about 5% to about 10% of the total weight of the composition.

15. The stable pharmaceutical composition according to claim 1, wherein the moisture sensitive active pharmaceutical ingredient has a major degradation product and wherein the composition comprises this major degradation product in an amount not more than about 3% by weight of the total initial weight of the moisture sensitive active pharmaceutical ingredient in the pharmaceutical composition after storage.

16. The stable pharmaceutical composition according to claim 15, where in the amount of the major degradation product of the moisture sensitive active pharmaceutical ingredient in the pharmaceutical composition is not more than about 2% by weight of the total initial weight of the moisture sensitive active pharmaceutical ingredient.

17. The stable pharmaceutical composition according to claim 16, where in the amount of the major degradation product of the moisture sensitive active pharmaceutical ingredient in the pharmaceutical composition is not more than about 1% by weight of the total initial weight of the moisture sensitive active pharmaceutical ingredient.

18. The stable pharmaceutical composition according to claim 15, wherein storage is in a package with moisture barrier properties, which are at least as efficient as aluminum-aluminum cold form blisters.

19. The stable pharmaceutical composition according to claim 18, wherein storage is at 55° C. for four weeks.

20. The stable pharmaceutical composition according to claim 1, wherein the composition is in a solid dosage form.

21. The stable pharmaceutical composition according to claim 20, wherein the dosage form is selected from the group consisting of a tablet and a capsule.

22. The stable pharmaceutical composition according to claim 21, wherein the dosage form is a tablet.

23. The stable pharmaceutical composition according to claim 22, wherein the tablet comprises a cosmetic tablet coating.

24. The stable pharmaceutical composition according to claim 23, wherein the cosmetic tablet coating has moisture barrier properties.

25. The stable pharmaceutical composition according to claim 24, wherein the cosmetic tablet coating having moisture barrier properties is selected from the group consisting of the Opadry® 85F series tablet coatings.

26. The stable pharmaceutical composition according to claim 23, wherein the cosmetic tablet coating is in an amount of about 2% to about 6% of the tablet weight.

27. The stable pharmaceutical composition according to claim 26, wherein the amount of the cosmetic tablet coating is about 3% to about 3.5% of the tablet weight.

28. A method of preparing a pharmaceutical composition comprising a moisture sensitive active pharmaceutical ingredient and a second active pharmaceutical ingredient comprising the following steps of:
a) providing a moisture sensitive active pharmaceutical ingredient;
b) mixing the moisture sensitive active pharmaceutical ingredient with at least one pharmaceutically acceptable excipient, forming a mixture; and
c) wet granulating the mixture with a solution of a binder excipient dissolved in one or more processing solvents forming a wet granulate;
d) providing a material comprising a second active pharmaceutical ingredient and optionally one or more pharmaceutical excipients; and
e) adding the material from step d) to the wet granulate from step c) forming a combined granulate,
wherein when the material of step d) comprises a second pharmaceutical ingredient and one or more pharmaceutical excipients the material is optionally a mixture obtained by mixing the second pharmaceutical ingredient with the one or more pharmaceutical excipients.

29. The method according to claim 28, wherein the amount of the moisture sensitive active pharmaceutical ingredient is about 0.1% to about 25% and the amount of the second active pharmaceutical ingredient is about 1% to about 25% of the total weight of the composition.

30. The method according to claim 29, wherein the amount of the moisture sensitive active pharmaceutical ingredient is about 0.6% to about 2.7% and the amount of the second active pharmaceutical ingredient is about 5% to about 10% of the total weight of the composition.

31. The method according to claim 28, wherein the moisture sensitive active pharmaceutical ingredient is Cilazapril and the second pharmaceutical ingredient is Hydrochlorothiazide.

32. The method according to claim 28, wherein the binder is selected from the group consisting of cellulose derivatives, polyvinyl pyrrolidones and their derivatives, polyvinyl acetates, and polyvinyl alcohols.

33. The method according to claim 32, wherein the binder is selected from the group consisting of Copovidone and Hypromellose.

34. The method according to claim 28, wherein the amount of the binder is at least about 4% of the total weight of the composition.

35. The method according to claim 34, wherein the amount of the binder is about 5% to about 10% of the total weight of the composition.

36. The method according to claim 28, wherein the processing solvent is selected from the group consisting of ethanol, isopropanol, water, and combinations thereof.

37. The method according to claim 28, wherein the binder is applied as a solution in water or ethanol.

38. The method according to claim 37, wherein the solution of the binder in water or ethanol comprises about 25% to about 55% (w/w) of the binder.

39. The method according to claim 38, wherein the solution of the binder in water or ethanol comprises about 30% to about 50% (w/w) of the binder.

40. The method according to claim 28, wherein the method further comprises the steps of
f) mixing the combined granulate with one or more excipients forming a final blend;
g) pressing the final blend into a tablet; and
h) optionally coating the tablet with a cosmetic coat.

41. The method according to claim 40, wherein the step of coating the tablet comprises preparing a suspension comprising about 10% to about 15% of a powder mixture for cosmetic coating, and applying the suspension on the tablet.

42. The method according to claim 41, wherein the suspension comprises about 12% to about 13% of a powder mixture for cosmetic coating.

43. The method according to claim 41, wherein the cosmetic coat has moisture barrier properties and the powder mixture for cosmetic coating is selected from the powder mixtures of the Opadry® 85F series.

44. The method according to claim 28 in preparing a pharmaceutical composition, wherein the method further comprises mixing the granulate with one or more excipients forming a final blend and filling the final blend in a capsule.

45. A method of treating a patient suffering from a disease comprising administering to a patient in need thereof a therapeutically effective amount of a stable pharmaceutical composition comprising a moisture sensitive active pharmaceutical ingredient, a second active pharmaceutical ingredient, and at least one pharmaceutical excipient, wherein the active pharmaceutical ingredients are wet granulated with a solution of the at least one pharmaceutical excipient, and wherein the moisture sensitive active pharmaceutical ingredient is first wet granulated with a solution of at least one pharmaceutical excipient in a processing solvent not containing the second active pharmaceutical ingredient before granulation with the second active pharmaceutical ingredient.

46. The method according to claim 45, wherein the disease is hypertension.

47. The method according to claim 45, wherein the moisture sensitive active pharmaceutical ingredient is Cilazapril, the second active pharmaceutical ingredient is Hydrochlorothiazide, and at least one pharmaceutical excipient is a binder.

48. The method according to claim 47, wherein the binder is selected from the group consisting of Copovidone and Hypromellose.
